(19) United States
(12) Patent Application Publication
(10) Pub. No.: US 2025/0255793 A1
(43) Pub. Date: Aug. 14, 2025

(54) ANTI-AGING CATALYST COMPOSITION COMPRISING A BLEND OF COSMETIC ACIDS

(71) Applicant: LOREAL, Paris (FR)

(72) Inventors: Rabije SAHITI, Lodi, NJ (US); Xue LIU, Berkeley Heights, NJ (US); Xi YAN, Berkeley Heights, NJ (US); Maggie SU, Cranford, NJ (US); Stephen LYNCH, Westfield, NJ (US); I-Chien LIAO, Princeton, NJ (US)

(73) Assignee: L'OREAL, Paris (FR)

(21) Appl. No.: 19/049,035

(22) Filed: Feb. 10, 2025

Related U.S. Application Data
(60) Provisional application No. 63/551,731, filed on Feb. 9, 2024.
(60) Provisional application No. 63/551,736, filed on Feb. 9, 2024.

(30) Foreign Application Priority Data
Apr. 18, 2024 (FR) 2404035
Apr. 19, 2024 (FR) 2404067

(51) Int. Cl. A61K 8/44; A61K 8/365; A61K 8/368; A61K 8/55; A61K 8/67; A61Q 19/08

(52) U.S. Cl. CPC A61K 8/447; A61K 8/365; A61K 8/368; A61K 8/55; A61K 8/676; A61K 8/678; A61Q 19/08; A61K 2800/5922; A61K 2800/74; A61K 2800/87

(57) ABSTRACT

An anti-aging catalyst composition includes a combination of cosmetically acceptable acids including at least three alpha hydroxy acids, at least one beta hydroxy acid, and one or more other cosmetically acceptable acids, the total amount of cosmetically acceptable acids in the anti-aging catalyst composition present in a range from about 5% to about 70%, each one of the cosmetically acceptable acids in the anti-aging catalyst composition present in a range from about 0.1% to about 69%, wherein the -aging catalyst composition has a pH ranging from about pH 1 to about pH 5.5.

### RELATED APPLICATIONS

This application claims the benefit of and priority to U.S. Provisional Patent Application No. 63/551,731 filed on Feb. 9, 2024, U.S. Provisional Patent Application No. 63/551,736 filed on Feb. 9, 2024, French Application No. 2404035 filed on Apr. 18, 2024 and French Application No. 2404067 filed on Apr. 19, 2024, the disclosures of which are hereby incorporated herein by reference in their entireties.

### FIELD OF THE INVENTION

The present invention is generally directed to an anti-aging catalyst composition that is used as a catalyst prior to use of skincare anti-aging compositions. The antiaging catalyst enhances skin anti-aging benefits of any anti-aging skincare routines as demonstrated by the improvement of skin properties, especially on the face, for example in reducing forehead wrinkles and fine lines, providing enhanced radiance all as compared with use of the anti-aging skin care composition or treatment regimen alone.

### BACKGROUND OF THE INVENTION

There are a variety of skin conditions that benefit from cosmetic products that may be applied to skin to brighten skin, reduce one or more of fine lines on the face, dullness, dryness, tightness, dark spots, and reduce the effects of acne or inflammatory lesions, enhance desquamation and skin renewal, and activate new cell proliferation in the skin, among other possible benefits. Many skin care products provide active ingredients that address one or more of these or other skin issues, yet there are challenges in providing products that have sufficiently high amounts of actives to confer the desired benefits.

Accordingly, there is a need in the art for a composition that can augment the effectiveness of anti-aging skin care products, particularly composition and treatment regimens for modulating, ameliorating, reducing, or reversing the manifestation of the dermatological signs of chronologically aged, hormonally aged, or photo-aged skin.

The inventors have provided an anti-aging catalyst composition that is pre-applied to skin in advance of an anti-aging skin care composition or treatment regimen. The anti-aging catalyst composition has demonstrated, through in vitro tests, the ability to regulate the expression of genes linked to skin aging. In certain embodiments, this composition may contribute to the improvement of tissue desquamation, epidermal development, cell cycle, and cell division to thereby confer improved skin quality. Compared to the anti-aging skin care composition or treatment regimen alone, the anti-aging catalyst composition used prior to application of an anti-aging skin care composition or treatment regimen has been shown statistically and clinically by the inventors to improve visible fine lines and radiance immediately after the initial application, and confer a statistically significant reduction in forehead wrinkle/fine line count at week 8.

### SUMMARY OF THE INVENTION

The summary is provided to introduce a selection of concepts in a simplified form that are further described below in the detailed description of the invention. This summary is not intended to identify key features of the claimed subject matter, nor is it intended to be used as an aid in determining the scope of the claimed subject matter.

In various embodiments, the invention that includes an anti-aging catalyst composition comprising: a blend of cosmetically acceptable acids comprising at least one beta hydroxy acid and at least three alpha-hydroxy acids, and one or more other cosmetically acceptable acid; and a cosmetically acceptable solvent comprising at least water, wherein the pH of the skin care composition is in a range from about pH 3.6 to about pH 3.9.

In an embodiment, the invention provides an anti-aging catalyst composition comprising: a blend of cosmetically acceptable acids comprising at least one beta hydroxy acid and at least three alpha-hydroxy acids, and one or more other cosmetically acceptable acids; and a cosmetically acceptable solvent comprising at least water, wherein the pH of the skin care composition is in a range from about pH 3.6 to about pH 3.9, wherein each one of the acids in the blend of acids is present in an amount effective to positively affect one or more of cornification, epidermis development, and keratinization, and wherein the skin care composition affects one or more of modulation, amelioration, reduction, or reversal of dermatological signs of chronologically aged, hormonally-aged, or photo-aged skin.

In some embodiments, each one of the cosmetically acceptable acids in the anti-aging catalyst composition is present in a range from about 0.1% to about 69%, and wherein the -aging catalyst composition has a pH ranging from about pH 1 to about pH 5.5.

In some embodiments, the total amount of cosmetically acceptable acid present is in a range from about 0.5% to about 70%.

In some embodiments, the blend of cosmetically acceptable acids includes at least salicylic acid, glycolic acid, lactic acid, mandelic acid and phytic acid, and wherein the one or more other cosmetically acceptable acids that is not an alpha- or beta-hydroxy acid includes at least phytic acid.

In some embodiments, the anti-aging catalyst composition comprises at least one amino acid comprising at least taurine.

In some embodiments, the blend of cosmetically acceptable acids that includes at least salicylic acid, glycolic acid, lactic acid, mandelic acid and phytic acid, the blend of cosmetically acceptable acids present in the composition in a total amount of 7.7%, by weight, based on the total weight of anti-aging catalyst composition.

In some embodiments, each one of the acids is present in the blend of acids in an amount effective to: statistically significantly improve visible fine lines and radiance immediately after the initial application, and when applied to skin prior to application of an anti-aging skin care composition or treatment regimen, confer a statistically significant reduction in forehead wrinkle/fine line count at week 8 as compared with use of the anti-aging skin care composition or treatment regimen alone; up-regulate the expression level of at least one gene associated with regulating cell division and at least one gene associated with promoting epidermal differentiation, and down-regulate the expression level of at least one gene associated with cornification, epidermis development, and keratinization, in skin to which the anti-aging catalyst composition is applied followed by application of a skin-care composition comprising at least ascorbic acid or a derivative thereof, ferulic acid and tocopherol, and wherein the skin care composition affects modulation, amelioration, reduction, or reversal of dermatological signs of chronologically aged, hormonally-aged, or photo-aged skin; stimulate an increase in one or more of Kallikrein gene in skin to which the anti-aging catalyst composition is applied followed by application of a skin-care composition comprising at least ascorbic acid or a derivative thereof, ferulic acid and tocopherol; or combinations thereof.

In some embodiments, the anti-aging catalyst composition includes any one or combination of pH adjusters, chelating agents, skin actives, humectants, skin penetrating agents, antioxidants, plant extracts, plant oils and butters, fragrances, fillers, powders, clays, pearlescent agents, odor absorbers, coloring materials and dyes, essential oils, vitamins, antimicrobials and preservatives, nature based oils, synthetic oils, silicone oils, hydrocarbon based oils, waxes, surfactants, emulsifiers, or a combination thereof.

In some embodiments, the anti-aging catalyst composition excludes any one or combination of pH adjusters, chelating agents, skin actives, humectants, skin penetrating agents, antioxidants, plant extracts, plant oils and butters, fragrances, fillers, powders, clays, pearlescent agents, odor absorbers, coloring materials and dyes, essential oils, vitamins, antimicrobials and preservatives, nature based oils, synthetic oils, silicone oils, hydrocarbon based oils, waxes, surfactants, emulsifiers, or a combination thereof.

In some embodiments, the anti-aging catalyst composition excludes any one or combination of ascorbic acid and other forms of vitamin C, ferulic acid, gallic acid, zinc PCA, azelaic acid, chlorogenic acid, and vitamin E, parabens, phthalates, cyclomethicones, silicone, mineral oil, petrol based ingredients, synthetic dyes, sulfates, polyquaternium, microplastics, EDTA, silicone oils, mineral UV filter agents, organic UV filter agents, or a combination thereof.

In some embodiments, the anti-aging catalyst composition is in the form of a serum, emulsion, or gel.

In an embodiment, the invention provides an anti-aging catalyst composition comprising: a blend of cosmetically acceptable acids comprising at least one beta hydroxy acid and at least three alpha-hydroxy acids, and one or more other cosmetically acceptable acids, wherein the blend of cosmetically acceptable acids includes at least salicylic acid, glycolic acid, lactic acid, mandelic acid and phytic acid, the blend of cosmetically acceptable acids present in the composition in a total amount of 7.7%, by weight, based on the total weight of anti-aging catalyst composition; at least one amino acid comprising taurine, and a cosmetically acceptable solvent comprising at least water, wherein the pH of the skin care composition is in a range from about pH 3.6 to about pH 3.9.

In an embodiment, the invention provides a method of modulating, ameliorating, reducing, or reversing the manifestation of the dermatological signs of chronologically aged, hormonally aged, or photo-aged skin, comprising: administering to the skin a cosmetically, dermatologically, pharmaceutically, or physiologically effective amount of the anti-aging catalyst composition that includes an anti-aging catalyst composition comprising: a blend of cosmetically acceptable acids comprising at least one beta hydroxy acid and at least three alpha-hydroxy acids, and one or more other cosmetically acceptable acid; and a cosmetically acceptable solvent comprising at least water, wherein the pH of the skin care composition is in a range from about pH 3.6 to about pH 3.9; the effective amount including an amount sufficient to: up-regulate the expression level of at least one gene associated with regulating cell division and at least one gene associated with promoting epidermal differentiation; down-regulate the expression level of at least one gene associated with cornification, epidermis development, and keratinization; or a combination thereof.

In some embodiments, the treatment with the anti-aging catalyst composition is followed by treatment of the skin with an anti-aging skin care composition or treatment regimen that includes one or more composition ingredients selected from the group consisting of antioxidants, Vitamin C, vitamin E, retinol, niacinamide, panthenol, glycerin, squalane, ceramides, collagen, hyaluronic acid, peptides, plant and fruit extracts, and sun filters, and combinations thereof.

In some embodiments, the anti-aging skin care composition or treatment regimen includes a skin care composition comprising at least ascorbic acid or a derivative thereof, ferulic acid and tocopherol, and the anti-aging catalyst composition includes taurine, and the blend of cosmetically acceptable acids in the anti-aging catalyst composition includes at least salicylic acid, glycolic acid, lactic acid, mandelic acid and phytic acid, the blend of cosmetically acceptable acids present in the composition in a total amount of 7.7%, by weight, based on the total weight of anti-aging catalyst composition.

This disclosure describes exemplary embodiments in accordance with the general inventive concepts and is not intended to limit the scope of the invention in any way. Indeed, the invention as described in the specification is broader than and unlimited by the exemplary embodiments set forth herein, and the terms used herein have their full ordinary meaning.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows on the left a bar graph showing results of evaluation of differentially expressed genes (“DEGs”) in tissue treated with vitamin C, vitamin E and ferulic acid treatment composition (“CEF”), and on the right principal component analysis (PCA) results in treatments with and without CEF;

FIG. 2 shows on the left variance map of DEGs in tissue treated with CEF, and on the right associated GO enrichment analysis;

FIG. 3 shows on the left a bar graph showing results of evaluation of DEGs in tissue treated with CEF+an embodiment of the inventive anti-aging catalyst (“Catalyst”), and on the right principal component analysis (PCA) results in treatments with CEF+Catalyst, and without either CEF or Catalyst;

FIG. 4 shows on the left variance map of DEGs in tissue treated with CEF+Catalyst, and on the right associated GO enrichment analysis;

FIG. 5 shows data in three charts relating to specific genes up- and down-regulated under control (no treatment), treated with CEF, or treated with CEF+Catalyst;

FIG. 6 shows data in three charts relating to specific genes up- and down-regulated under control (no treatment), or treated with CEF;

FIG. 7 shows data in two charts relating to specific genes up- and down-regulated under control (no treatment), or treated with CEF, and a graphical image of generally understood cell cycle for understanding the table on the left;

FIG. 8 shows data in three charts relating to specific genes up- and down-regulated under control (no treatment), or treated with CEF+Catalyst;

FIG. 9 shows clinical study data relating to treatments with CEF+Catalyst; and

FIG. 10 shows Kallikrein gene expression results under control (no treatment), treatment with CEF alone, treatment with Catalyst (Catalyst Peel) alone, or treated with CEF+Catalyst.

### DETAILED DESCRIPTION OF THE INVENTION

In some embodiments, the anti-aging catalyst composition is in the form of a serum which may exclude oils, surfactants, and emulsifiers. In some embodiments, the anti-aging catalyst composition is in the form of an emulsion and thus comprises an oil phase that includes one or more oils and may include one or more surfactants or emulsifiers or combinations thereof. In some embodiments, the anti-aging catalyst composition is in the form of a gel.

In the context of the invention, a serum refers to a hydrophilic liquid composition formulated for topical application. A serum may optionally be free from or essentially free from one or more of an oil, including an emollient, a wax, a surfactant, an emulsifier, a silicone oil, or a combination or all of the foregoing.

The terms “Exclude, “Free” and “Essentially Free” mean that, while it is preferred that no excluded material is present in the anti-aging catalyst composition, it is possible to have very small amounts of the excluded material in the anti-aging catalyst composition of the invention, provided that these amounts do not materially affect the advantageous properties of the anti-aging catalyst composition. In particular, “essentially free” means that excluded material can be present in the anti-aging catalyst composition at an amount of less than 5%, or less than 4%, or less than 3%, or less than 2%, or less than 1%, or less than 0.1% by weight, or 0%, based on the total weight of the anti-aging catalyst composition.

In some embodiments, the anti-aging catalyst composition is free or essentially free of at least one active selected from the group consisting of ascorbic acid and other forms of vitamin C, ferulic acid, gallic acid, zinc PCA, azelaic acid, chlorogenic acid, and vitamin E, or a combination thereof.

In some embodiments, the anti-aging catalyst composition lacks or minimizes use of non-renewable ingredients (e.g., mineral oil and petrol based ingredients) and ingredients that have negative environmental impact (e.g., EDTA that mobilizes heavy metals). Thus, in some embodiments anti-aging catalyst composition is free or essentially free ingredients selected from the group consisting of parabens, phthalates, cyclomethicones, silicone, mineral oil, petrol based ingredients, synthetic dyes, sulfates, polyquaternium, microplastics, EDTA, silicone oils, mineral UV filter agents, organic UV filter agents, or a combination thereof.

When paired with anti-aging routines, the antiaging catalyst composition is able to enhance skin anti-aging benefits as illustrated by the improvement in forehead wrinkles and fine lines vs. the routines by themselves. Active ingredients in the anti-aging routines paired with antiaging catalyst include but are not limited to: antioxidants (including Vitamins C, E, and their derivatives), chemical peels, retinols, niacinamide, panthenol, glycerin, squalane, ceramides, collagen, hyaluronic acid, peptides, plant and fruit extracts, and sun filters.

When paired with anti-aging routines, for example, but not limited to antioxidant treatments (AOX), AHA peels, retinol products, the antiaging catalyst may enhance radiance immediately post-application as compared to the anti-aging routines by themselves.

When paired with antioxidant formulations, the antiaging catalyst may enhance the AOX protection compared to the formulation alone.

The usage of the anti-aging skin care composition may include application once a day, at morning or night, after cleansing and before treatment with an anti-aging skin care composition or regimen. The anti-aging skin care composition can be used all year round. Application includes a soaked cotton pad (1 dropper) or directly by hand (8-10 drops). After application of the anti-aging skin care composition, the skin care regimen can be applied anywhere from immediate to 6 hrs later.

The anti-aging skin care composition can be paired with home-use anti-aging devices (e.g., red light-based devices).

Accordingly, in various embodiments, the anti-aging catalyst composition is provided according to the following detailed description.

### Cosmetically Acceptable Acids (Acid Based Skin Actives)

The anti-aging catalyst composition includes a combination of cosmetically acceptable acids, the combination of cosmetically acceptable acids including a combination of at least three alpha hydroxy acids, at least one beta hydroxy acid, and one or more other cosmetically acceptable acids. In some embodiments, the anti-aging catalyst composition includes at least lactic acid, glycolic acid, mandelic acid, salicylic acid, and phytic acid. In some particular embodiments, the anti-aging catalyst composition includes only lactic acid, glycolic acid, mandelic acid, salicylic acid, and phytic acid.

The anti-aging catalyst composition is a water based serum form that may be a rinse off or a leave on formulation, wherein the anti-aging catalyst composition has a viscosity that is flowable when applied. In some embodiments, the anti-aging catalyst composition may be provided infused into a suitable substrate or contained in a dispenser.

In various embodiments, the total amount of cosmetically acceptable acid present in a range from about 1% to about 70%, or from about 5% to about 70%, or from about 5% to about 20%, or from about 5% to about 10%, or from about 5% to about 8%, by weight, based on the total weight of anti-aging catalyst composition, and, as needed, a neutralizing agent may be present to provide a pH in a range from about pH 3.6 to about pH 9, or from about pH 1 to about pH 5.5, and in some embodiments about pH 3.5 to pH 3.9. In some particular embodiments, the anti-aging catalyst composition has a pH of 3.7.

In various embodiments, when present, the neutralizing agent is present in a range from about 0.1% to about 5%, by weight, based on the total weight of anti-aging catalyst composition.

### Alpha Hydroxy Acids

The anti-aging catalyst composition comprises at least three alpha hydroxy acids. In some particular embodiment, the at least three alpha hydroxy acids include glycolic acid, lactic acid and mandelic acid.

Suitable alpha hydroxy acids include glycolic acid, lactic acid, mandelic acid, tartaric acid, citric acid, ester derivatives thereof, or a combination thereof. Exemplary ester derivatives include ester compounds of lactic acid, such as methyl lactate, ethyl lactate, butyl lactate and, similarly, ester compounds of glycolic acid, tartaric acid, mandelic acid, citric acid. One particularly suitable alpha hydroxy acid is lactic acid. Lactic acid, or 2-hydroxypropanoic acid, is provided composition to provide enhanced exfoliation of the skin. In addition, lactic acid also boosts production of glycosaminoglycan (GAG) in the skin, improving the barrier function and moisturization of skin.

The anti-aging catalyst composition may include a total concentration of alpha hydroxy acid in a range from about 0.3% to about 69%, or from about 0.3% to about 20%, or from about 1% to about 15%, or from about 2.5% to about 6%, or is about 5.5%, or any suitable combination, sub-combination, range, or sub-range thereof by weight, based on the weight of the anti-aging catalyst composition. One of ordinary skill in the art, however, will appreciate that other ranges are within the scope of the invention. In some embodiments, the total amount of alpha hydroxy acid present is not more than about 10%, or not more than about 6%. In various embodiments, each one of the cosmetically acceptable acids in the anti-aging catalyst composition is present in a range from about 0.1% to about 69%, wherein the -aging catalyst composition has a pH ranging from about pH 1 to about pH 5.5.

In some particular embodiments, alpha hydroxy acid is present in a range from about 0.1% to about 5% and in some embodiments is present at about 2.5%, lactic acid is present in a range from about 0.1% to about 5% and in some embodiments is present at about 2%, and mandelic acid is present in a range from about 0.1% to about 5% and in some embodiment is present at about 1% by weight of the anti-aging catalyst composition.

In some embodiments, the total amount of cosmetically acceptable alpha hydroxy acids is present in a range from about 0.3% to about 15% and in some embodiments is about 5.5% by weight of the anti-aging catalyst composition.

Thus, any one of or a combination of alpha hydroxy acid when present, may be present, by weight, based on the total weight of anti-aging catalyst composition, from about 0.1, 0.2, 0.3, 0.4, 0.5, 0.6, 0.7, 0.8, 0.9, 1.0, 1.2, 1.3, 1.4, 1.5, 1.6, 1.7, 1.8, 1.9, 2, 3, 4, 5, 6, 7, 8, 9, 10, 11, 12, 13, 14, to about 15 or up to about 69%, by weight, based on the weight of the anti-aging catalyst composition, including increments and ranges therein and there between.

### Beta Hydroxy Acids

In The anti-aging catalyst composition comprises at least one beta hydroxy acid. In some particular embodiments, the anti-aging catalyst composition may include a beta hydroxy acid comprising salicylic acid. In some particular embodiments, the anti-aging catalyst composition includes only one beta hydroxy acid comprising salicylic acid.

The term “beta-hydroxy acid” is understood to mean, according to the present invention, a carboxylic acid having a hydroxyl functional group and a carboxylic functional group separated by two carbon atoms. A beta hydroxy acid can be present in the anti-aging catalyst composition in the form of the free acid and/or in the form of one of its associated salts (salts with an organic base or an alkali metal, in particular), especially according to the final pH imposed on anti-aging catalyst composition.

Suitable beta hydroxy acids include salicylic acid and derivatives thereof (including 5-n-octanoylsalicylic acid, salicylate, sodium salicylate, and willow extract), capryloyl salicylic acid, beta hydroxybutanoic acid, propionic acid, beta-hydroxy beata-methylbutyric acid, carnitine tropic acid, and trethocanic acid, and combinations of these.

And in some particular embodiments, the beta hydroxy acid in the anti-aging catalyst composition may include at least one of salicylic acid and derivatives thereof (including 5-n-octanoylsalicylic acid, salicylate, sodium salicylate, and willow extract), capryloyl salicylic acid, beta hydroxybutanoic acid, propionic acid, beta-hydroxy beata-methylbutyric acid, carnitine tropic acid, and trethocanic acid.

The anti-aging catalyst composition includes a concentration of beta hydroxy acid in a range from about 0.1% up to and not more than about 2% of beta hydroxy acid, by weight, based on the total weight of anti-aging catalyst composition. In some embodiments, the anti-aging catalyst composition may include up to and not more than about 2%, or about 1.9% of beta hydroxy acid. In some embodiments, the anti-aging catalyst composition may include up to and not more than about 1% of beta hydroxy acid. In some embodiments, the amount of beta hydroxy acid, if present is not more than about 0.40% to about 0.50%.

In some embodiments, the anti-aging catalyst composition may include from about 0.1% to about 1% of beta hydroxy acid, or from about 0.2% to about 2.0%, or from about 0.1% to about 1.5%, or from about 0.2% to about 1.5%, or from about 0.3% to about 1.0%, or from about 0.35% to about 0.75%, or from about 0.4% to about 0.5%, or is about 0.45%, or any suitable combination, sub-combination, range, or sub-range thereof by weight, based on the weight of the anti-aging catalyst composition. One of ordinary skill in the art, however, will appreciate that other ranges are within the scope of the invention.

Thus, the at least one beta hydroxy acid, is present in the anti-aging catalyst composition from about 0.1, 0.2, 0.3, 0.4, 0.5, 0.6, 0.7, 0.8, 0.9, 1.0, 1.2, 1.3, 1.4, 1.5, 1.6, 1.7, 1.8, 1.9, to about 2.0 or up to about 69%, by weight, based on the weight of the anti-aging catalyst composition, including increments and all ranges and subranges therein and there between.

### Other Acids; Phytic Acid

The anti-aging catalyst composition includes one or more other cosmetically acceptable acids that are not alpha- or beta-hydroxy acids.

In some embodiments, the anti-aging catalyst composition may include of phytic acid. Phytic acid is also known as phytate and is a six-fold dihydrogenphosphate ester of inositol (specifically, of the myo isomer), also called inositol hexakisphosphate (IP6) or inositol polyphosphate. At physiological pH, the phosphates are partially ionized, resulting in the phytate anion. Phytic acid is found naturally in plant seeds and is a storage form of phosphorus.

Phytic acid may be present in the anti-aging catalyst composition in an amount from about 0.5% to about 69%, or from about 0.5% to about 15%, or from about 0.5% to about 5%, and in some embodiments, from about 1% to about 4%, and in some embodiments, from about 1.5% to about 2.5%, or about 2%, or any suitable combination, sub-combination, range, or sub-range thereof by weight, based on the weight of the anti-aging catalyst composition. As exemplified in the instant disclosure, the phytic acid raw material may be provided at a dilution of 50% or as otherwise disclosed herein.

Thus, in various embodiments, phytic when present, may be present in the anti-aging catalyst composition according to the disclosure, and each of the individual components in the ranges as described herein above, from about 0.50, 0.60, 0.70, 0.80, 0.90, 1.0, 2, 3, 4, 5, 6, 7, 8, 9, 10, 11, 12, 13, 14, to about 15 percent or up to about 69%, by weight, based on the weight of the anti-aging catalyst composition, including increments and ranges there between.

### Amino Acid

The anti-aging catalyst composition may include at least amino acid. In some embodiments the at least one amino acid may be selected from the group consisting of naturally occurring amino acids. In some embodiments the at least one amino acid comprises taurine.

As non-limiting examples, the amino acids that may be used may be of natural or synthetic origin, in L, D, or racemic form, and comprise at least one acid function chosen from, for instance, carboxylic acid, sulfonic acid, phosphonic acid, and phosphoric acid functions. The amino acids may be in their neutral or ionic form. Polymeric forms are also useful, such as poly arginine, poly lysine, etc. Additional non-limiting examples include basic amino acids comprising an additional amine function optionally included in a ring or in a ureido function. In some cases, amino acids that may be used include, but are not limited to, taurine, aspartic acid, glutamic acid, alanine, arginine, ornithine, citrulline, asparagine, carnitine, cysteine, glutamine, glycine, histidine, lysine, isoleucine, leucine, methionine, N-phenylalanine, proline, serine, threonine, tryptophan, tyrosine, ornithine, citrulline, and valine.

In accordance with the various embodiments, the amount of amnio acid, for example, taurine, present in a composition according to the disclosure can range from about 0.1% to about 10%, or from about 0.2% to about 9%, or from about 0.5% to about 8%, or from about 0.8% to about 2%, or about 1%, or any suitable combination, sub-combination, range, or sub-range thereof by weight, based on the weight of the composition. In some embodiments according to the disclosure, the composition includes at least about 0.5%, or at least about 1% of the at least one amino acid. In some embodiments according to the disclosure, the composition includes not more than about 1% of the at least one amino acid. One of ordinary skill in the art, however, will appreciate that other ranges are within the scope of the invention.

Thus, any one of or a combination of the amino acid for example, taurine, may be present, by weight, based on the total weight of the composition, or from about 0.1, 0.2, 0.3, 0.4, 0.5, 0.6, 0.7, 0.8, 0.9, 1.0, 1.2, 1.3, 1.4, 1.5, 1.6, 1.7, 1.8, 1.9, 2.0, 2.5, 3.0, 3.5, 4.0, 4.5, 5.0, 5.5, 6.0, 6.5, 7.0, 7.5, 8.0, 8.5, 9.0, 9.5 to about 10.0 percent, by weight, based on the weight of the anti-aging catalyst composition, including increments and ranges therein and there between.

### Solvents

The anti-aging catalyst composition includes water and optionally one or more water soluble solvents.

In accordance with the various embodiments, water is present in the anti-aging catalyst composition in a range from about 10% to about 99%, or from about 30% to about 95%, or from about 50% to about 90%, or from about 75% to about 85%, or any suitable combination, sub-combination, range, or sub-range thereof by weight, based on the weight of the anti-aging catalyst composition. One of ordinary skill in the art, however, will appreciate that other ranges are within the scope of the invention.

Thus, water may be present by weight, based on the weight of anti-aging catalyst composition, from about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 75, 80, 85, 90, 91, 92, 93, 94, 95, 96, 97, 98 to about 99 weight percent, including increments and ranges therein and there between.

The water used may be sterile demineralized water and/or a floral water such as rose water, cornflower water, chamomile water or lime water, and/or a natural thermal or mineral water such as, for example: water from Vittel, water from the Vichy basin, water from Uriage, water from La Roche Posay, water from La Bourboule, water from Enghien-les-Bains, water from Saint Gervais-les-Bains, water from Neris-les-Bains, water from Allevar-les-Bains, water from Digne, water from Maizieres, water from Neyrac-les-Bains, water from Lons-le-saunier, water from Eaux Bonnes, water from Rochefort, water from Saint Christau, water from Les Fumades, water from Tercis-les-Bains or water from Avene. The water phase may also comprise reconstituted thermal water, that is to say a water comprising trace elements such as zinc, copper, magnesium, etc., reconstituting the characteristics of a thermal water.

The pH of anti-aging catalyst composition is adjusted based on the amount of cosmetically acceptable acid to be in a range as stated herein above. The pH is adjusted to the desired value by addition of a base (organic or inorganic), for example sodium hydroxide, potassium hydroxide, or another suitable base, or a combination thereof.

### Water-Soluble Solvents

In some embodiments, the anti-aging catalyst composition may include at least one water-soluble solvent. The term “water-soluble solvent” is interchangeable with the term “water-miscible solvent” and means a compound that is liquid at 25° C. and at atmospheric pressure (760 mmHg), and it has a solubility of at least 50% in water under these conditions. In some cases, the water-soluble solvent has a solubility of at least 60%, 70%, 80%, or 90% in water under these conditions. Non-limiting examples of water-soluble solvents include, for example, glycerin, alcohols (for example, selected from water-soluble C1-C30, C1-C15, C1-C10, or C1-C4 alcohols), organic solvents, polyols, glycols, or mixtures thereof.

In some particular embodiments according to the disclosure, when present, a water-soluble solvent may include glycerin.

As examples of organic solvents, non-limiting mentions can be made of the following organic solvents: ethyl alcohol, isopropyl alcohol, propyl alcohol, benzyl alcohol, and phenylethyl alcohol, or glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol. Other suitable examples of organic solvents are ethylene glycol, propylene glycol, butylene glycol, hexylene glycol, propane diol, and glycerin. The organic solvents can be volatile or non-volatile compounds.

Further non-limiting examples of water-soluble solvents include alkanols (polyhydric alcohols such as glycols and polyols) such as glycerin, 1,2,6-hexanetriol, trimethylolpropane, ethylene glycol, propylene glycol, diethylene glycol, butylene glycol, hexylene glycol, triethylene glycol, tetraethylene glycol, pentaethylene glycol, dipropylene glycol, 1,3-butanediol, 2,3-butanediol, 1,4-butanediol, 3-methyl-1,3-butanediol, 1,5-pentanediol, tetraethylene glycol, 1,6-hexanediol, 2-methyl-2,4-pentanediol, polyethylene glycol, 1,2,4-butanetriol, 1,2,6-hexanetriol, 2-butene-1,4-diol, 2-ethyl-1,3-hexanediol, 2-methyl-2,4-pentanediol, (caprylyl glycol), 1,2-hexanediol, 1,2-pentanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, or mixtures thereof.

In accordance with the various embodiments the amount of the at least one water-soluble solvent, when present, is from about 0.1% to about 25%, or from about 0.1% to about 2%, or from about 0.1% to about 1%, or from about 0.1% to about 0.8%, or from about 0.1% to about 0.5%, or from about 1% to about 20%, or from about 1% to about 10%, or from about 2% to about 8%, or any suitable combination, sub-combination, range, or sub-range thereof by weight, based on the weight of the anti-aging catalyst composition. One of ordinary skill in the art, however, will appreciate that other ranges are within the scope of the invention. In some embodiments, the anti-aging catalyst composition includes more than one water soluble solvent, each water soluble solvent present in an amount as set forth herein above, wherein each different water soluble solvent may be present within one of the ranges selected from the ranges set forth herein above.

Thus, each one or combination of water-soluble solvents, when present, may be present by weight, based on the total weight of anti-aging catalyst composition, from about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24 to about 25 percent, by weight, based on the weight of the anti-aging catalyst composition, including increments and ranges therein and there between.

### Other Ingredients

In some embodiments, there may be one or more optional actives or other ingredients (herein, “additives”) present in the anti-aging catalyst composition.

In some embodiments, the anti-aging catalyst composition may include additional ingredients, including, but not limited to, pH adjusters, chelating agents, skin actives, humectants, skin penetrating agents, antioxidants, plant extracts, plant oils and butters, fragrances, fillers, powders, clays, pearlescent agents, odor absorbers, coloring materials and dyes, essential oils, vitamins, antimicrobials and preservatives, oils (nature based and synthetic, including but not limited to silicone oils, hydrocarbon based oils, and waxes), as well as surfactants, emulsifiers, and combinations of these. Non limiting examples of some of these optional additional ingredients and others are provided herein below.

In some embodiments, the anti-aging catalyst composition excludes any one or combination of pH adjusters, chelating agents, skin actives, humectants, skin penetrating agents, antioxidants, plant extracts, plant oils and butters, fragrances, fillers, powders, clays, pearlescent agents, odor absorbers, coloring materials and dyes, essential oils, vitamins, antimicrobials and preservatives, oils (nature based and synthetic, including but not limited to silicone oils, hydrocarbon based oils, and waxes), as well as surfactants, emulsifiers, or a combination thereof.

In some embodiments, the anti-aging catalyst composition may include ingredients selected from: chelating agents, such as, trisodium ethylenediamine disuccinate, ethylenediaminetetraacetic acid (EDTA), tetrasodium glutamate diacetate, tetrasodium etidronate, tetrasodium pyrophosphate, pentasodium ethylenediamine tetramethylene phosphonate, sodium staminate and combinations of these; humectants, such as acetamide MEA; dihydroxy and trihydroxy alcohols, such as glycerin, ethoxydiglycol, propylene glycol, propanediol, butylene glycol, dipropylene glycol, pentylene glycol, hexylene glycol, or a combination thereof; anti-microbials; antioxidants, including, but not limited to, phenolic compounds, such as chalcones, flavones, flavanones, flavanols, flavonols, dihydroflavonols, isoflavonoids, neoflavonoids, catechins, anthocyanidins, tannins, lignans, aurones, stilbenoids, curcuminoids, alkylphenols, betacyanins, capsacinoids, hydroxybenzoketones, methoxyphenols, naphthoquinones, and phenolic terpenes, resveratrol, curcumin, pinoresinol, ferulic acid, hydroxytyrosol, cinnamic acid, caffeic acid, p-coumaric acid, baicalin (Scutellaria Baicalensis root extract), pine bark extract (Pinus Pinaster bark/bud extract), ellagic acid; hyaluronic acid and its derivatives; escin (also known as Aescin, a mixture of saponins with anti-inflammatory, vasoconstrictor and vasoprotective effects found in Aesculus hippocastanum; hydroxyacetophenone and 4-hydroxyacetophenone; and vitamins and vitamin derivatives, such as vitamin A, vitamin C (I-ascorbic acid, acetyl-C (3-O-Ethyl ascorbic acid), magnesium ascorbyl phosphate, sodium ascorbyl phosphate, tetrahexyldecyl ascorbate, ascorbylglucoside, or a combination thereof), vitamin E (tocopherol, alpha-tocopherol, beta-tocopherol, delta-tocopherol, gamma-tocopherol, and alpha-tocotrienol, beta-tocotrienol, delta-tocotrienol, gamma-tocotrienol, and derivatives thereof), vitamin D, calcium pantothenate; piroctone olamine; phenylethyl resorcinol; escin; retinol; citric acid; hydroxypropyl tetrahydropyrantriol; sequestrants; niacinamide, pyruvic acid; UV filters and boosters; dispersants; ceramides; opacifiers; or a combination thereof.

In some embodiments, the one or more additives may be selected from fillers and/or clays of mineral or organic origin, natural or synthetic in nature in order to provide oil absorption or optical effects. Oil absorption fillers may impart a matte effect and non-greasy feeling onto the skin. Optical effects fillers may impart a soft-focus/haze/blur effect to the skin, provide the skin with a more uniform appearance, reduce the appearance of skin imperfections or discoloration, or reduce the visibility of pores. A filler may include boron nitride, methyl methacrylate crosspolymer, mica, zea may (corn) starch, magnesium oxide, nylon-12, nylon-66, cellulose, polyethylene, talc, talc (and) methicone, talc (and) dimethicone, perlite, sodium silicate, pumice, PTFE, Ammonium Polyacryloyldimethyl Taurate, polymethyl methacrylate, Oryza sativa (rice) starch, aluminum starch octenylsuccinate, potato starch modified, alumina, calcium sodium borosilicate, magnesium carbonate, hydrated silica, dimethicone/vinyl dimethicone crosspolymer, sodium carboxymethyl starch, bismuth oxychloride, silica silylate, boron nitride, iron oxide, calcium carbonate, calcium sulfate (and) iron oxides, sodium potassium aluminum silicate, silica, silica (and) methicone, silica (and) dimethicone, polysilicone-22, polysilicone-8, polysilicone-11, methyl methacrylate crosspolymer, polymethylsilsesquioxane, methylsilanol/silicate crosspolymer, vinyl dimethicone/methicone silsesquioxane crosspolymer, diphenyl dimethicone/vinyl diphenyl dimethicone silsesquioxane crosspolymer, and styrene/acrylates copolymer, mica (and) lauroyl lysine, synthetic fluorphlogopite (and) titanium dioxide (and) magnesium silicate (and) tin oxide, synthetic fluorphlogopite (and) titanium dioxide (and) tin oxide, titanium dioxide, or a combination thereof. Clays may include smectite, sodium smectite, calcium smectite, illite, chlorite, vermiculite, attapulgite, phylosilicates, kaolinites, bentonite, hectorite, kaolin, silicates, magnesium aluminum silicate, montmorillonite (volcanic minerals), or a combination thereof.

In some embodiments, additives may include one or a combination of antimicrobial agents and their salts, for example, including, but not limited to, the group consisting of chlorphenesin, caprylyl glycol, phenoxyethanol, caprylhydroxamic acid, benzoic acid, salicylic acid, benzyl alcohol, phenethyl alcohol, benzalkonium chloride, 4-hydroxyacetophenone, piroctone olamine, hexyl glycerin, ethylhexylglycerin, octylglycerin, benzylglycerin, 3-heptoyl-2,2-propandiol, and 1,2-hexandiol, and pentylene glycol, or a combination thereof.

In some embodiments, the one or more additives may be selected from penetrants or penetration enhancers selected from Ethoxydiglycol, Dimethyl isosorbide, Sodium dilauramidoglutamide lysine, Polyglyceryl Fatty Acid Ester, Laurocapram, dipropylene glycol, propylene glycol, or a combination thereof.

In some embodiments, additives may include one or a combination of cosmetic oils. In some embodiments, the oil is generally immiscible in water. The oil may be selected from hydrocarbons, silicones, fatty alcohols, glycols, and vegetable oils. The oil may include one or a combination of polar and non-polar oil. In some embodiments, the oil may be chosen from hydrocarbon-based oils from plants or of plant origin, mineral oil, ester oils, fatty alcohols containing from 12 to 26 carbon atoms, fatty acids containing from 12 to 26 carbon atoms and vinylpyrrolidone copolymers, or a combination thereof.

In some embodiments, additives may include one or a combination of nonionic, cationic, anionic, or zwitterionic surfactant, or a combination thereof.

In some embodiments, additives may include one or a combination of thickeners, such as natural gums and synthetic polymers, for example, the thickener may be selected from the group consisting of starches (corn, rice, tapioca, potato), gums (xanthan carrageenan, gellan, sclerotium, tarabiotech fermentation), hydroxypropyl guar, ceratonia siliqua (carob) gum, acrylates/C10-30 alkyl acrylate crosspolymer, carbomer, ammonium polyacryloyldimethyl taurate, ammonium acryloyldimethyltaurate/steareth-25 methacrylate crosspolymer, and polyacrylate crosspolymer-6, or a combination thereof.

In some embodiments, additives may include one or a combination of alcohols, for example, mono-alcohols such as monohydric C1-C8 alcohols such as ethanol, propanol, butanol, isopropanol, isobutanol, and benzyl alcohol, and phenylethyl alcohol, or a combination thereof.

Although the optional additives are given as examples, it will be appreciated that other optional components compatible with cosmetic applications known in the art may be used that are suitable. It will be appreciated by a skilled artisan that any optional additives are present only to the extent and in amounts that do not materially adversely affect the basic and novel characteristic(s) of the claimed disclosure. Thus, in some embodiments that include optional additives, such optional additives will not materially adversely affect the solubility of the skin actives of the anti-aging catalyst composition. And in some embodiments that include optional additives, such optional additives will not materially adversely affect the anti-aging catalyst composition stability or activity.

For the avoidance of doubt, in some embodiments, the anti-aging catalyst composition excludes any one or combination of the foregoing listed additive and active components.

In some embodiments, the anti-aging catalyst composition is free or essentially free of at least one active selected from the group consisting of ascorbic acid and other forms of vitamin C, ferulic acid, gallic acid, zinc PCA, azelaic acid, chlorogenic acid, and vitamin E, or a combination thereof.

In some embodiments, the anti-aging catalyst composition lacks or minimizes use of non-renewable ingredients (e.g., mineral oil and petrol based ingredients) and ingredients that have negative environmental impact (e.g., EDTA that mobilizes heavy metals). Thus, in some embodiments anti-aging catalyst composition is free or essentially free ingredients selected from the group consisting of parabens, phthalates, cyclomethicones, silicone, mineral oil, petrol based ingredients, synthetic dyes, sulfates, polyquaternium, microplastics, EDTA, silicone oils, mineral UV filter agents, organic UV filter agents, or a combination thereof.

In accordance with the various embodiments, the amounts of additives, for example, actives and other components, when present in the anti-aging catalyst composition can range from about 0.001% to about 50%, from about 0.5% to about 30%, from about 1.5% to about 20%, or from about 5% to about 15%, or any suitable combination, sub-combination, range, or sub-range thereof by weight, based on the weight of the anti-aging catalyst composition.

In some embodiments, one or more additives, alone or in combination, can be present in the anti-aging catalyst composition from about 0.05% to about 50% by weight, from about 0.05% to about 25%, from about 0.1% to about 10%, from about 0.25% to about 5%, and from about 0.5% to about 3.5%, from about 0.05% to about 2.5% by weight, from about 0.1% to about 2%, from about 0.25% to about 1.5%, or from about 0.5% to about 1.25%, or any suitable combination, sub-combination, range, or sub-range thereof by weight, based on the weight of the anti-aging catalyst composition.

Thus, one or a combination of additives, when present, may be present in the anti-aging catalyst composition, by weight, based on the weight of the anti-aging catalyst composition, each one or the combination present from about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.10, 0.20, 0.30, 0.40, 0.50, 0.60, 0.70, 0 0.80, 0.90, 1.0, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 to about 50 weight percent, including increments and ranges therein and there between.

### EXAMPLES

### Example 1: Raw Materials

Percentages of each ingredient as may be exemplified in the anti-aging catalyst composition example embodiment are shown as amount of active ingredient, wherein the raw materials may be present in an amount that is equal to the amount of active, or if the raw material has a concentration of active that is less than 100%, the anti-aging catalyst composition includes the raw material that includes active and a suitable solvent, wherein the concentration of active in the raw material is provided herein below in Table 1. If a RM is not listed in the table, it should be presumed to have an active concentration that is essentially 100% active.

TABLE 1
Select Raw Materials (including RMs having
active concentrations of less than 100%)

| | | Percent active |
|---|---|---|
| Ingredient | Source | (if less than 100%) |
| GLYCOLIC | GUANGAN CHENGXIN | 70 |
| ACID | CHEMICAL, CHEMOURS | |
| LACTIC | CORBION PRODUTOS | 90 |
| ACID | RENOVAVEIS | |
| MANDELIC | KUMAR ORGANICS | |
| ACID | PRODUCTS LIMITED | |
| PHYTIC | TSUNO RICE FINE CHEMICALS | 50 |
| ACID | | |
| SALICYLIC | JQC (HUAYIN) | |
| ACID | PHARMACEUTICAL, ALTA | |
| | LABORATORIES, NOVACYL | |
| TAURINE | JIANGSU YUANYANG PHARM | |
| | SOLD QUIMDIS, QIANJIANG | |
| | YONGAN PHARMA | |

### Example 2: Inventive Anti-Aging Catalyst Composition

The following represents an exemplary embodiment of the inventive; other embodiments are possible consistent with the description set forth herein above.

TABLE 2
Inventive Anti-Aging Catalyst Composition

| | INV 1 |
|---|---|
| | Antiaging |
| Ingredient | Catalyst |
| CHLORPHENESIN | 0.20 |
| TAURINE | 1.0 |
| CAPRYLYL GLYCOL | 0.20 |
| ETHOXYDIGLYCOL | 2.40 |
| SODIUM HYDROXIDE | 1.75 |
| TETRASODIUM GLUTAMATE DIACETATE | 0.048 |
| GLYCERIN | 3.00 |
| SALICYLIC ACID | 0.20 |
| WATER | 83.70 |
| GLYCOLIC ACID | 2.5 |
| LACTIC ACID | 2.0 |
| PHYTIC ACID | 2.00 |
| MANDELIC ACID | 1.00 |

Note:
amounts represent active ingredient final concentration, though some actives are provided in RM that have less than 100% active concentration

### Example 3: Taurine Induced Nad+ Stimulation

Home use micropeels are a convenient and effective way for individuals to perform gentle chemical peels in the comfort of their own homes. These skincare treatments involve the application of mild exfoliating solutions to help improve skin texture, clarity, and radiance without the need for professional intervention (See Yoshimura T, Manabe C, Inokuchi Y, Mutou C, Nagahama T, Murakami S. Protective effect of taurine on UVB-induced skin aging in hairless mice. Biomed Pharmacother. September 2021; 141:111898.). Home use micro peels are especially popular among those looking to address mild skin concerns and achieve a fresher, smoother complexion. In general, AHAs, such as glycolic acid and lactic acid, work to exfoliate the skin's outer layer, while BHAs, like salicylic acid, can penetrate deeper into the pores, making them suitable for addressing acne and clogged pores. The chemical solution gently dissolves dead skin cells, promoting cell turnover and stimulating the growth of new, healthier skin.

Vitamin C, vitamin E and ferulic acid is an advanced antioxidant serum. This potent formula combines 15% pure vitamin C (L-ascorbic acid), 1% vitamin E (alpha-tocopherol), and 0.5% ferulic acid. The unique blend of these ingredients provides superior protection against environmental stressors, such as UV radiation and pollution, which can cause premature aging and damage to the skin. The high concentration of vitamin C in vitamin C, vitamin E and ferulic acid has been scientifically proven to neutralize free radicals, stimulate collagen synthesis, and brighten the skin's tone. Regular use of vitamin C, vitamin E and ferulic acid has been clinically shown to improve skin texture, reduce the appearance of fine lines and wrinkles, and promote a more even and radiant complexion.

The in vitro testing described herein examines an embodiment of the anti-aging catalyst (micropeel), vitamin C, vitamin E and ferulic acid and the combination of Anti-aging catalyst composition with vitamin C, vitamin E and ferulic acid formula using reconstructed skin. Anti-aging catalyst composition can maximize the outcomes, while amplifying benefits, of skincare at home. The study described herein involves investigation into anti-aging catalyst composition micropeel, vitamin C, vitamin E and ferulic acid products and also the effects on pairing anti-aging catalyst composition with vitamin C, vitamin E and ferulic acid. Furthermore, anti-aging catalyst composition formula also included taurine as an active, which is hypothesized to supplement the function of anti-aging catalyst composition serum by boosting cell NAD+ level.

NAD+ plays an important role in cell energy metabolism, as it forms reduced NADH, which furnishes reducing equivalents to the mitochondrial electron transport chain (ETC) to fuel oxidative phosphorylation. The roles of NAD+, however, have expanded beyond its role as a coenzyme, as NAD+ and its metabolites also act as degradation substrates for a wide range of enzymes, such as the sirtuins. (See Canto C, Menzies K J, Auwerx J. NAD (+) Metabolism and the Control of Energy Homeostasis: A Balancing Act between Mitochondria and the Nucleus. Cell Metab. Jul. 7 2015; 22 (1): 31-53.) The role of NAD+ as a coenzyme in most metabolic pathways suggests that NAD+ limitations could affect metabolic efficiency. Decreasing NAD+ levels could therefore prompt the development of many of the ailments associated with aging. (Canto)

Taurine is one of the most abundant free amino acids in mammalian tissues and its concentration in intracellular spaces reaches the millimolar level in some cell types. In a previous study, oral taurine supplementation is able to suppress UVB-induced wrinkle formation, which may be associated with the regulation of moisture content in the epidermis. The beneficial effects of taurine on skin aging may be attributed to its osmoregulatory role. (See Yoshimura T, Manabe C, Inokuchi Y, Mutou C, Nagahama T, Murakami S. Protective effect of taurine on UVB-induced skin aging in hairless mice. Biomed Pharmacother. September 2021; 141:111898.) In addition, dietary taurine supplementation ameliorates MDA levels, GSSG/GSH, and NAD+/NADH in diabetic precataractous lens. (See Obrosova I G, Stevens MJ. Effect of dietary taurine supplementation on GSH and NAD (P)-redox status, lipid peroxidation, and energy metabolism in diabetic precataractous lens. Invest Ophthalmol Vis Sci. March 1999; 40 (3): 680-8.)

### Taurine Induced Nad+ Stimulation Results

Cell studies were conducted in which the effects of taurine and niacinamide on the NAD+ production were assessed when keratinocytes were cultured with these raw materials. In all of the tested conditions supplemented with actives, the NAD+ of the cells were increased compared to the untreated control. It was found that 0.2% niacinamide appeared to induce a higher level of NAD+ compared to 0.05% niacinamide. It was found that 0.05% taurine induced a comparable NAD+ level as compared to 0.2% niacinamide.

Taurine has been identified as an active of interest for skin care due to its potential benefits to boost cell energy. This report explores the effect of supplementing taurine with keratinocytes to understand its effect on increasing NAD+ level in the cells. Taurine is demonstrated with this report to have stimulatory effect in increasing the NAD+ level, and it is contemplated that further testing may demonstrate effects of taurine on cell metabolism. In our in vitro study, we observed that vitamin C, vitamin E and ferulic acid imparts significant benefits to the cell cycle and cell division. When combined with anti-aging catalyst composition, the treatment not only enhances skin renewal and resurfacing benefits but also maintains the positive effects on cell cycle and cell division. This is evident in the more pronounced impact on pathways related to skin cornification and epidermis development compared to single type treatment.

### Example 4: Tissue Morphology & RNA-Seq Analysis Studies

Tissue studies using 3D skin (full thickness skin tissue from Episkin) were undertaken in which the tissue was cultured then treated with anti-aging catalyst composition (topical 5 mg/cm2) or 1 min, 5 min or 10 min, followed by rinsing off 3 times with PBS. After that, the tissues were incubated without any treatment treated as Peel only, the tissues treated with vitamin C, vitamin E and ferulic acid (Pure, topical 5 mg/cm2). The incubation time with topical application was 72 hours. Tissue was evaluated after sectioning and staining for macroscopic and microscopic effects and RNA sequence analyses were conducted to evaluate RNA levels of specific genes in the treated tissue.

The inventors treated the 3D skin with four different conditions: control, vitamin C, vitamin E and ferulic acid-treated, anti-aging catalyst composition-treated, and a combination of vitamin C, vitamin E and ferulic acid and anti-aging catalyst composition treatment. Subsequently, we conducted a differential expression analysis and examined the biological pathways through Gene Ontology (GO) analysis.

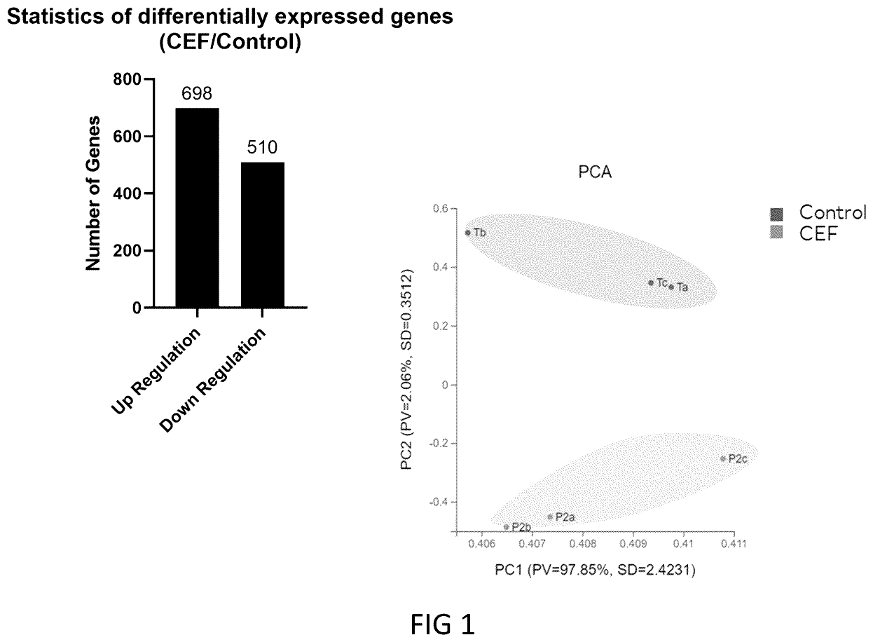

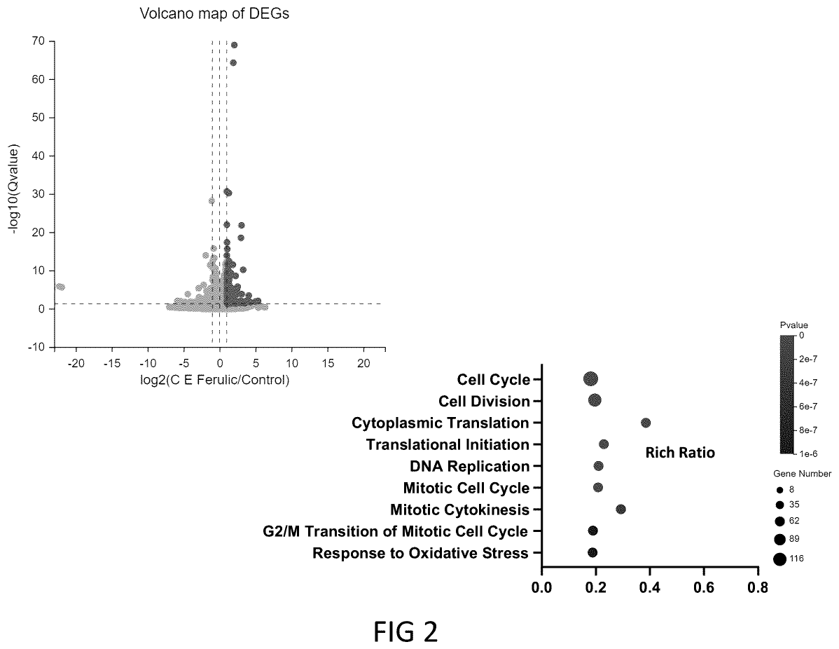

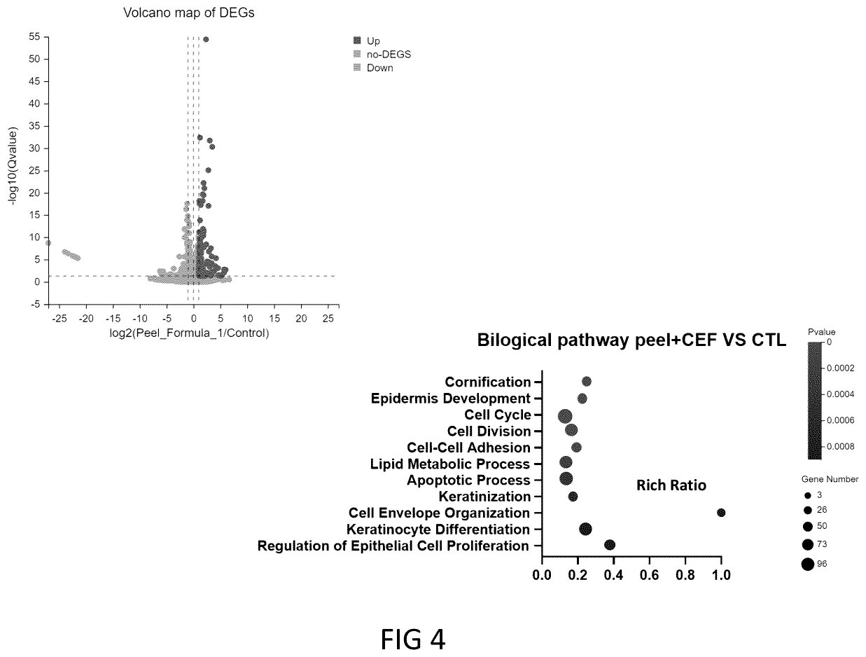

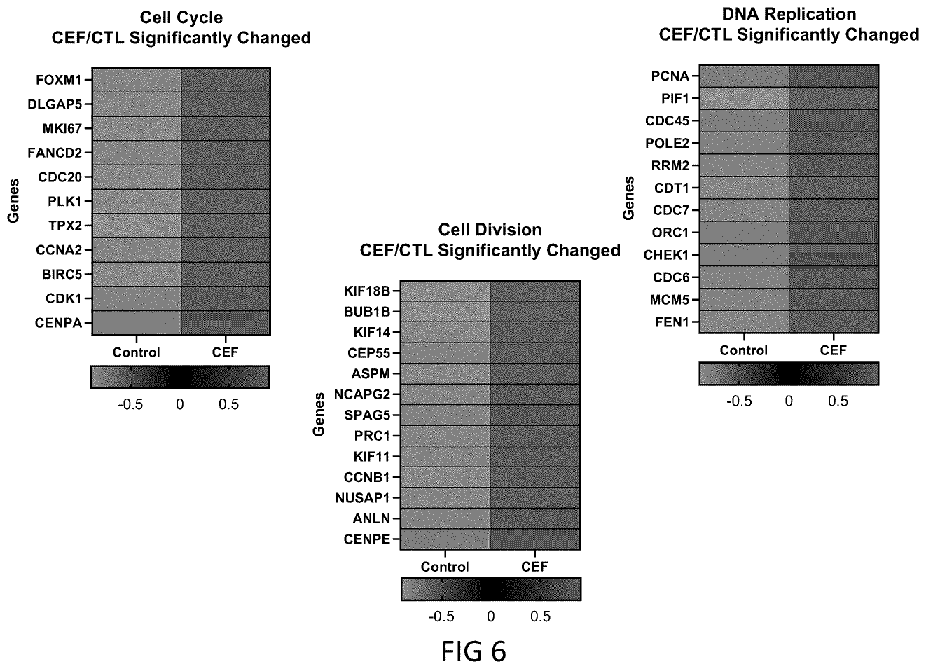

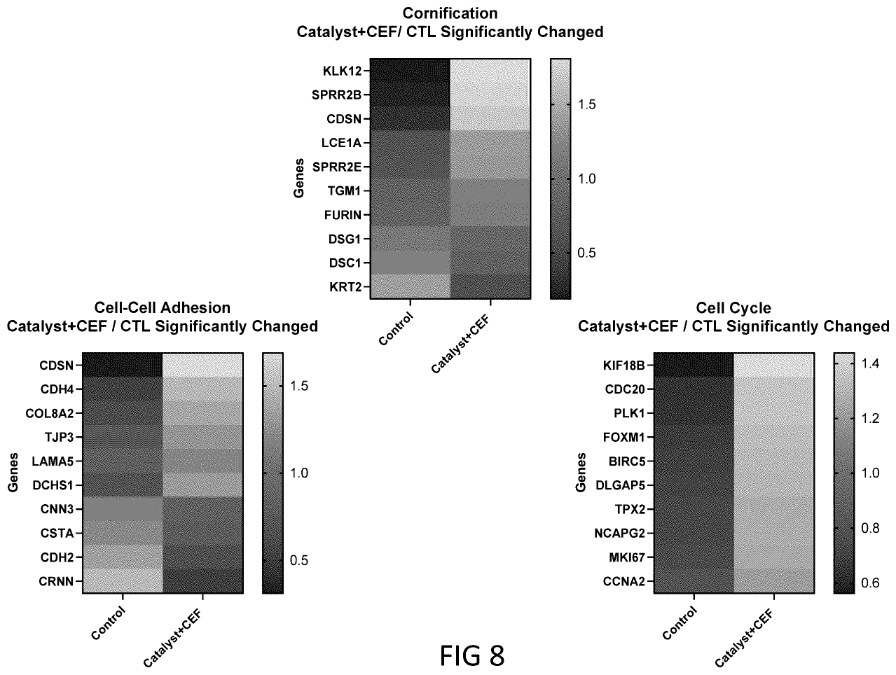

Referring to FIG. 1-8, to investigate the main functional pathway of DEGs (differentially expressed genes), GO enrichment analysis was carried out. Enrichment bubble diagrams of the GO biological process level are shown in FIG. 2 and FIG. 4, wherein the vertical axis represents the name of the pathway, the horizontal axis represents the rich factor, and the dot size indicates the number of differentially expressed genes in this pathway. The color of the points corresponds to different p value ranges. Rich factor refers to the ratio of the number of differential genes (sample number) enriched in the pathway to the number of annotation genes (background number). A greater Rich factor value indicates greater enrichment.

Referencing FIG. 1, principal component analysis (PCA) is widely used for data science and machine learning. It can tell us which few dimensions in the original coordinate dominate the data. First principal component (PC1) has the highest variance; the second principal component (PC2) has a second highest variance.

The studies revealed that compared to the control without treatment, vitamin C, vitamin E and ferulic acid treatment led to the upregulation of 698 genes and the downregulation of 510 genes. Anti-aging catalyst composition treatment alone resulted in the upregulation of 16 genes and the downregulation of 11 genes. Vitamin C, vitamin E and ferulic acid significantly altered 1208 genes compared to the control samples. The behavior of vitamin C, vitamin E and ferulic acid treated samples distinctly differed from untreated tissues, indicating significant effects. Referencing FIG. 2, out of all the genes that were significantly affected, the major biological pathways impacted include cell cycles, cell division, DNA replication, and other related pathways associated with the cell cycle.

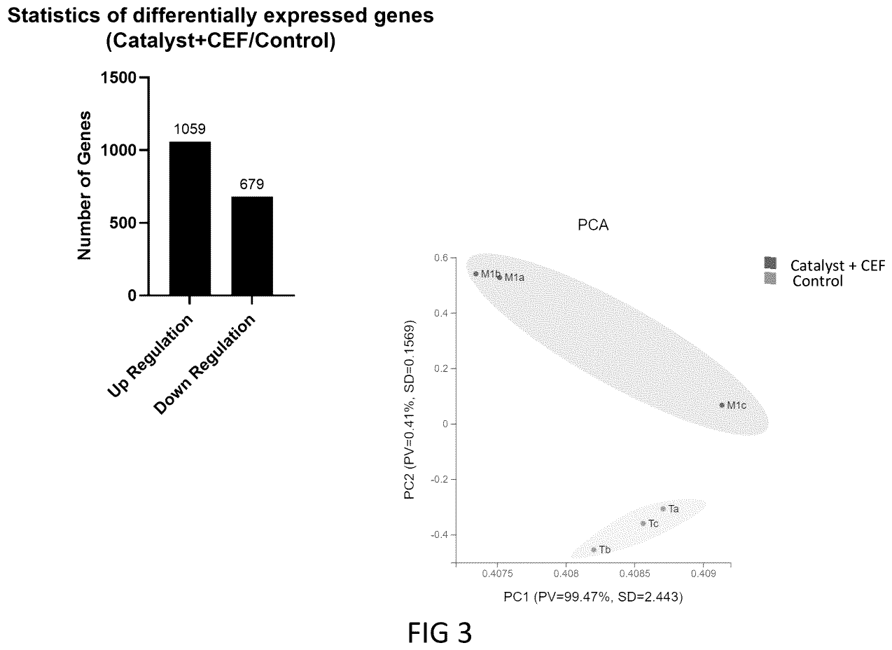

Referencing FIG. 3, notably, when vitamin C, vitamin E and ferulic acid was combined with anti-aging catalyst composition, we observed the upregulation of 1059 genes and the downregulation of 679 genes. Catalyst paired with vitamin C, vitamin E and ferulic acid significantly altered 1738 genes compared to the control samples. The behavior of Catalyst+vitamin C, vitamin E and ferulic acid treated samples distinctly differed from untreated tissues, indicating significant effects. Referencing FIG. 4, the pairing of Catalyst and CEF not only affects genes associated with cell cycle and cell division but also significantly impacts genes related to cornification, epidermis development, and keratinization.

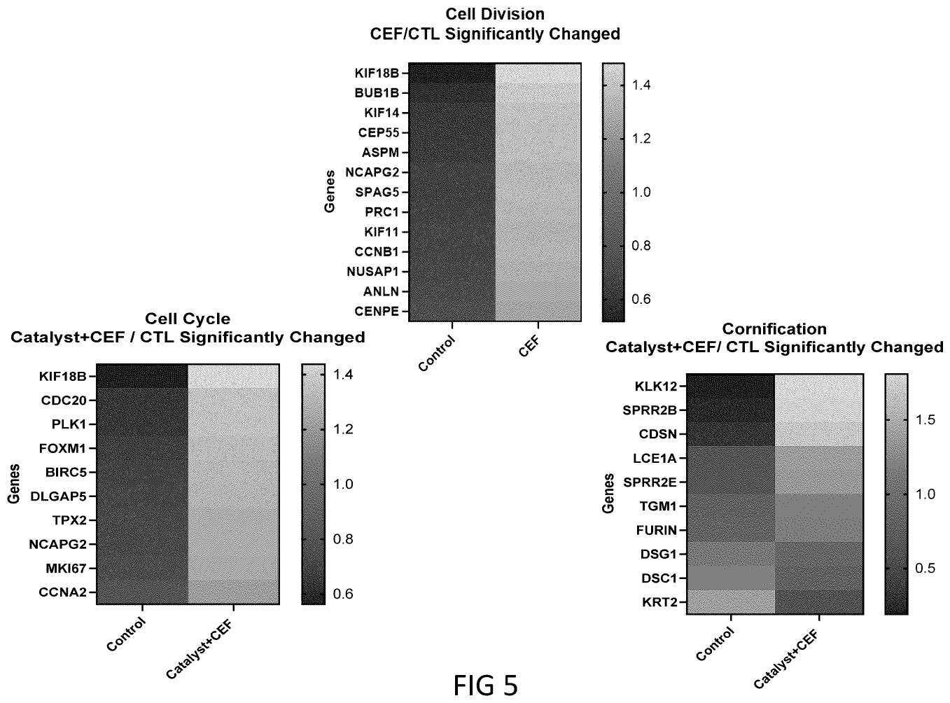

Referring again to the drawings, FIGS. 5-8, show results for vitamin C, vitamin E and ferulic acid (referenced in the drawings as “CEF” or control (“CTL”) as a treatment alone, and in combination with the inventive anti-aging catalyst composition (referenced in the drawings as “Catalyst”; the combination of Catalyst and CEF referred to as Catalyst+CEF) wherein the results demonstrate there are several genes, particularly in cell cycle and cell division pathways, that showed significant upregulation, and others with downregulation. A summary of their functions and effect of treatment with CEF alone or with catalyst is provided herein below and in the referenced drawings.

In the analyses of biological pathways, the inventors observed that cell cycle and cell division pathways were the most significantly altered with vitamin C, vitamin E and ferulic acid treatment, ranking as the top two impacted pathways, and DNA replication was also altered. Cornification and epidermis development pathways were also affected but appeared outside the top 20 pathways. In comparison, as demonstrated and further described herein, when tissues were treated with vitamin C, vitamin E and ferulic acid in combination with the inventive anti-aging catalyst composition, the order of pathway impact shifted, with cornification and epidermis development pathways taking precedence over cell cycle and cell division, and keratinization was also altered. This highlights a more pronounced effect of vitamin C, vitamin E and ferulic acid in combination with the inventive anti-aging catalyst composition in promoting tissue desquamation, epidermal development and turnover.

### Description of Functions of Genes Modulated by the Vitamin C, Vitamin E and Ferulic Acid or Anti-Aging Catalyst Composition+Vitamin C, Vitamin E and Ferulic Acid

### Vitamin C, Vitamin E and Ferulic Acid Upregulated:

KIF18B is a cell type-specific regulator of spindle orientation in the epidermis

BUB1B is a gene encodes a kinase involved in spindle checkpoint function.

KIF14 is essential for localizing citron kinase to the mitotic spindle.

CEP55 is essential for mitotic exit and cytokinesis.

ASPM is essential for maintaining orderly cell division.

NCAPG2 regulates mitotic chromosome architecture.

SPAG5 is involved in the functional and dynamic regulation of mitotic spindles.

PRC1 is a gene encodes a protein that is involved in cytokinesis.

KIF11 is a molecular motor protein that is essential in mitosis.

CCNB1 regulates transition of the cell from G2 to M phase.

NUSAP1 regulates basal cell migration through activation of the Hedgehog signaling pathway.

ANLN gene encodes an actin-binding protein that plays a role in cell growth and migration, and in cytokinesis.

CENPE is a kinesin-like motor protein that accumulates in the G2 phase of the cell cycle.

### Anti-Aging Catalyst Composition+Vitamin C, Vitamin E and Ferulic Acid Upregulated:

KIF18B is a cell type-specific regulator of spindle orientation in the epidermis.

CDC20 act as a regulatory protein interacting with many other proteins at multiple points in the cell cycle.

PLK1 plays multiple critical roles in centrosome maturation, mitotic chromosome segregation & cytokinesis.

FOXM1 controls the expression of genes required for both G1/S and G2/M transition

BIRC5 is an inhibitor of apoptosis (IAP) gene family, which encode negative regulatory proteins that prevent apoptotic cell death.

DLGAP5 is involved in several processes, including centrosome localization; kinetochore assembly; and mitotic spindle organization.

TPX2 is involved in mitotic spindle assembly during late prophase and early prometaphase.

NCAPG2 is essential participant of the condensin II complex involved in the process of chromosome cohesion and stabilization in mitosis.

MKI67 regulates chromosome segregation and regulation of mitotic nuclear division.

CCNA2 promotes transition through G1/S and G2/M.

KLK12 conserves homeostasis of the skin barrier through their roles in desquamation, antimicrobial defense, innate immune response, and barrier maintenance.

SPRR2B encodes for cross-linked envelope protein of keratinocytes.

CDSN plays a vital role in the structural and functional integrity of the epidermis and the hair follicle integrity.

LCE1A belongs to the late cornified envelope (LCE) gene cluster within the epidermal differentiation complex (EDC).

SPRR2E is a gene encoding for cross-linked envelope protein of keratinocytes.

TGM1 gene encodes for a catalytic membrane-bound enzyme that functions in the formation of the epidermal cornified cell envelope.

### Anti-Aging Catalyst Composition+Vitamin C, Vitamin E and Ferulic Acid Downregulated:

DSG1 is expressed everywhere in the skin epidermis, supports keratinocyte differentiation and suprabasal morphogenesis.

DSC1 maintains the structure of epidermis through its adhesive function.

KRT2 is involved in maintenance of cellular integrity, regulation of cell growth and migration, and protection from apoptosis.

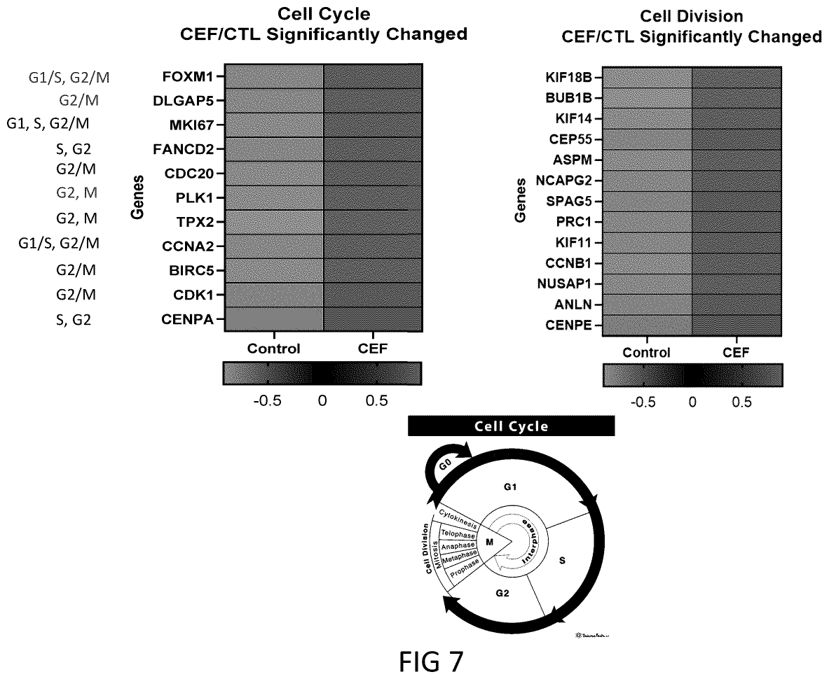

Referring now to FIG. 7, in the left panel, the drawing shows genes associated with the cell cycle in which expression is significantly changed when the cells are treated with CEF. Referencing the listed genes associated with the cell cycle, “/” (slash): The slash represents a transition or progression from one phase to another. For example, “G1/S” indicates the transition from the G1 phase to the S phase of the cell cycle, where DNA synthesis occurs. It signifies that the gene is active or relevant during the transition between those phases; “,” (comma) is used to separate different phases or stages of the cell cycle that are relevant to the gene. For example, “G2/M” indicates that the gene is active or relevant during both the G2 phase and the subsequent M phase (mitosis) of the cell cycle.

Referring again to FIG. 7, in the right panel, the following genes are identified as being understood to have the described features/functions in the general bioscience arts.

KIF18B: Involved in metaphase and anaphase.

BUB1B: Primarily associated with mitotic checkpoint control during metaphase and anaphase.

KIF14: Plays a role in cytokinesis, specifically in the formation and function of the midbody during late cytokinesis.

CEP55: Involved in cytokinesis and plays a role in the formation and function of the midbody during late cytokinesis.

ASPM: Associated with cytokinesis and plays a role in regulating cell division during late mitosis.

NCAPG2: Involved in multiple stages of mitosis, including prophase, prometaphase, metaphase, and anaphase.

SPAG5: Plays a role in spindle organization and chromosome alignment during prophase, prometaphase, and metaphase.

PRC1: Primarily associated with cytokinesis, particularly in the formation and function of the central spindle during late cytokinesis.

KIF11: Involved in multiple stages of mitosis, including prophase, prometaphase, metaphase, and anaphase.

CCNB1: Associated with multiple stages of the cell cycle, including the G2/M transition and the metaphase to anaphase transition during mitosis.

NUSAP1: Primarily associated with mitotic spindle assembly during prophase, prometaphase, and metaphase.

ANLN: Involved in cytokinesis and plays a role in the formation and function of the contractile ring during late cytokinesis.

CENPE: Plays a role in chromosome alignment and segregation during metaphase and anaphase.

It will be appreciated that while the above described genes have generally understood roles in specific stages of cell division, their functions may extend beyond those mentioned or may be influenced by specific conditions and cell types, including unexpected involvement in skin properties and response to compositions such as those disclosed herein.

### Example 5: Clinical Study

A split-face study was conducted in which clinical subjects were treated to different treatments, one on each side of the face involving treatment once daily over a period of 8 weeks. The control side included treatment with an anti-aging skincare routine involving application of the vitamin C, vitamin E and ferulic acid composition as describe herein above, and the test side included the same skin care regimen treatment plus treatment with the inventive cell cycle catalyst. Data were collected at time points including start (Baseline) and weeks 1, 4 and 8. Skin was evaluated for clinical grading, VISIA images, 3D Pimos imaging and analysis, and SAQs/Tolerance.

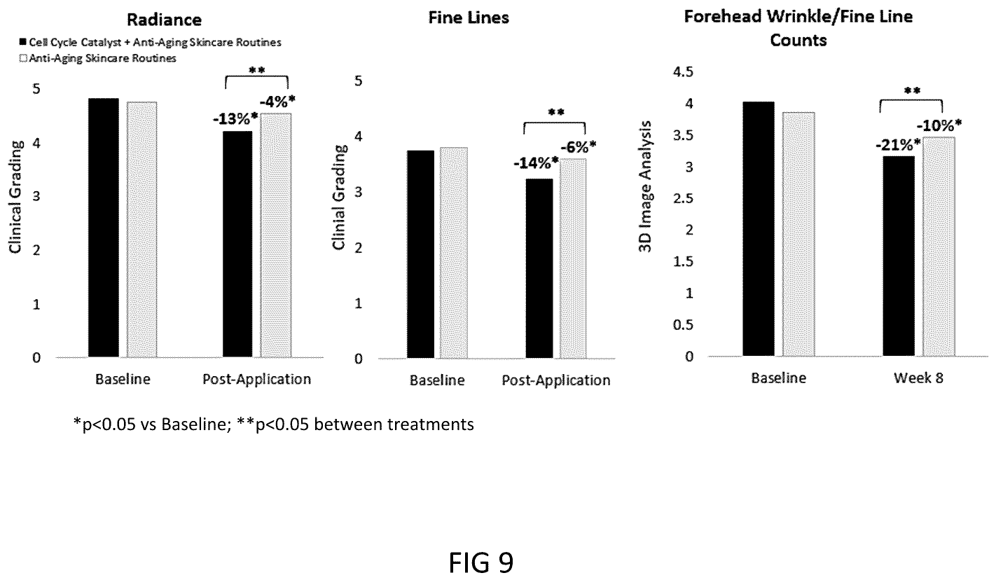

Referencing FIG. 9, in the clinical study, compared to anti-aging skincare routine alone, Cell Cycle Catalyst+anti-aging skincare routine showed clinically significant superiority in improving visible fine lines and radiance after the initial application, and a statistically significant reduction in forehead wrinkle/fine line count at week 8.

### Overall Study Results

As shown herein, the inventors have provided anti-aging catalyst composition that is exemplified herein to unexpectedly confer one or more benefits including, but not limited to: when applied to skin prior to application of an anti-aging skin care composition or treatment regimen, conferring a statistically significant improving visible fine lines and radiance immediately after the initial application; reducing forehead wrinkle/fine line count at week 8 as compared with use of the anti-aging skin care composition or treatment regimen alone, as described herein.

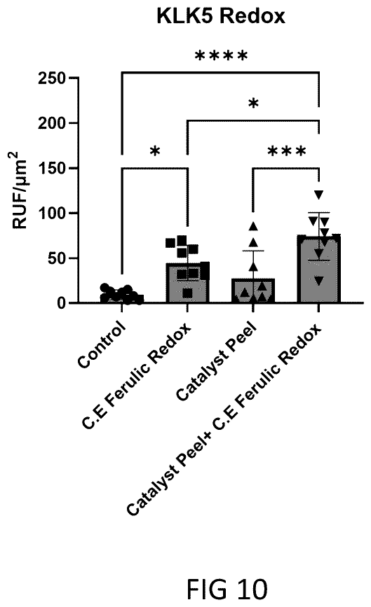

The anti-aging catalyst composition that is exemplified herein also unexpectedly confers one or more benefits including, but not limited to: when applied to skin prior to application of an anti-aging skin care composition or treatment regimen, up-regulating the expression level of at least one gene associated with regulating cell division and at least one gene associated with promoting epidermal differentiation, and down-regulating the expression level of at least one gene associated with cornification, epidermis development, and keratinization, and wherein the skin care composition effects modulation, amelioration, reduction, or reversal of dermatological signs of chronologically aged, hormonally-aged, or photo-aged skin; and stimulate an increase in Kallikrein one or more of gene expression in skin to which the anti-aging catalyst composition is applied followed by application of a skin-care composition comprising at least ascorbic acid or a derivative thereof, ferulic acid, and tocopherol, as compared to the skin-care composition alone (see FIG. 10).

When paired with an anti-aging skin care composition that includes at least vitamin C, vitamin E and ferulic acid (a vitamin C, vitamin E and ferulic acid formulation), the antiaging catalyst composition is able to enhance skin renewing and resurfacing benefits as illustrated by its more pronounced impact on the skin cornification, epidermis development function pathways compared to single vitamin C, vitamin E and ferulic acid treatment.

The inventors have showcased, through nucleic acid analyses of 3D reconstructed full-thickness skin treatments, the capacity of the antiaging catalyst composition to significantly enhance skin renewing and resurfacing benefits. In comparison to using a singular anti-aging skincare composition or treatment regimen alone, the antiaging catalyst composition notably amplifies the impact on skin cornification and epidermis development function pathways. Importantly, while other treatments also demonstrated these benefits, the antiaging catalyst composition showcased a heightened and distinctive efficacy, emphasizing its particular significance in augmenting these crucial pathways. This is evidenced by the upregulation of expression levels in specific genes associated with regulating cell division and promoting epidermal differentiation, as described herein. Furthermore, the results indicate a downregulation effect on the expression levels of genes associated with cornification, epidermis development, and keratinization.

The articles “a” and “an,” as used herein, mean one or more when applied to any feature in embodiments of the present invention described in the specification and claims. The use of “a” and “an” does not limit the meaning to a single feature unless such a limit is specifically stated. The article “the” preceding singular or plural nouns or noun phrases denotes a particular specified feature or particular specified features and may have a singular or plural connotation depending upon the context in which it is used. The adjective “any” means one, some, or all indiscriminately of whatever quantity.

“At least one,” as used herein, means one or more and thus includes individual components as well as mixtures/combinations.

The transitional terms “comprising,” “consisting essentially of,” and “consisting of,” when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step, or material. The term “consisting of” excludes any element, step, or material other than those specified in the claim and, in the latter instance, impurities ordinary associated with the specified material(s). The term “consisting essentially of” limits the scope of a claim to the specified elements, steps, or material(s) and those that do not materially affect the basic and novel characteristic(s) of the claimed invention. All materials and methods described herein that embody the present invention can, in alternate embodiments, be more specifically defined by any of the transitional terms “comprising,” “consisting essentially of,” and “consisting of.”

Other than in the operating examples, or where otherwise indicated, all numbers expressing quantities of ingredients and/or reaction conditions preceded by the word “about” are to be understood as being modified in all instances as meaning within 10% of the indicated number (e.g., “about 10%” means 9%-11% and “about 2%” means 1.8%-2.2%).

All percentages and ratios are calculated by weight unless otherwise indicated. All percentages are calculated based on the total composition unless otherwise indicated. Generally, unless otherwise expressly stated herein, “weight” or “amount” as used herein with respect to the percent amount of an ingredient refers to the amount of the raw material comprising the ingredient, wherein the raw material may be described herein to comprise less than and up to 100% activity of the ingredient. Therefore, weight percent of an active in a composition is represented as the amount of raw material containing the active that is used, and may or may not reflect the final percentage of the active, wherein the final percentage of the active is dependent on the weight percent of active in the raw material.

All ranges and amounts given herein are intended to include subranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. Further, it is understood that when an amount of a component is given, it is intended to signify the amount of the active material unless otherwise specifically stated.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements. The example that follows serves to illustrate embodiments of the present disclosure without, however, being limiting in nature.

While the invention has been described with reference to a preferred embodiment, it will be understood by those skilled in the art that various changes may be made, and equivalents may be substituted for elements thereof without departing from the scope of the invention. In addition, many modifications may be made to adapt a particular situation or material to the teachings of the invention without departing from the essential scope thereof. Therefore, it is intended that the invention is not limited to the particular embodiment disclosed as the best mode contemplated for carrying out this invention, but that the invention will include all embodiments falling within the scope of the appended claims.

What is claimed is:

1. An anti-aging catalyst composition comprising: a blend of cosmetically acceptable acids comprising at least one beta hydroxy acid and at least three alpha-hydroxy acids, and one or more other cosmetically acceptable acids; and a cosmetically acceptable solvent comprising at least water, wherein the pH of the skin care composition is in a range from about pH 3.6 to about pH 3.9, wherein each one of the acids in the blend of acids is present in an amount effective to positively affect one or more of cornification, epidermis development, and keratinization, and wherein the skin care composition affects one or more of modulation, amelioration, reduction, or reversal of dermatological signs of chronologically aged, hormonally-aged, or photo-aged skin.

2. The anti-aging catalyst composition according to claim 1, wherein each one of the cosmetically acceptable acids in the anti-aging catalyst composition is present in a range from about 0.1% to about 69%, and wherein the -aging catalyst composition has a pH ranging from about pH 1 to about pH 5.5.

3. The anti-aging catalyst composition according to claim 1, wherein the total amount of cosmetically acceptable acid present is in a range from about 0.5% to about 70%.

4. The anti-aging catalyst composition according to claim 1, wherein the blend of cosmetically acceptable acids includes at least salicylic acid, glycolic acid, lactic acid, mandelic acid and phytic acid, and wherein the one or more other cosmetically acceptable acids that is not an alpha- or beta-hydroxy acid includes at least phytic acid.

5. The anti-aging catalyst composition according to claim 1, comprising at least one amino acid comprising at least taurine.

6. The anti-aging catalyst composition according to claim 4, wherein the blend of cosmetically acceptable acids that includes at least salicylic acid, glycolic acid, lactic acid, mandelic acid and phytic acid, the blend of cosmetically acceptable acids present in the composition in a total amount of 7.7%, by weight, based on the total weight of anti-aging catalyst composition.

7. The anti-aging catalyst composition according to claim 1, wherein each one of the acids is present in the blend of acids in an amount effective to:
i. statistically significantly improve visible fine lines and radiance immediately after the initial application, and when applied to skin prior to application of an anti-aging skin care composition or treatment regimen, confer a statistically significant reduction in forehead wrinkle/fine line count at week 8 as compared with use of the anti-aging skin care composition or treatment regimen alone;
ii. up-regulate the expression level of at least one gene associated with regulating cell division and at least one gene associated with promoting epidermal differentiation, and down-regulate the expression level of at least one gene associated with cornification, epidermis development, and keratinization, in skin to which the anti-aging catalyst composition is applied followed by application of a skin-care composition comprising at least ascorbic acid or a derivative thereof, ferulic acid and tocopherol, and wherein the skin care composition affects modulation, amelioration, reduction, or reversal of dermatological signs of chronologically aged, hormonally-aged, or photo-aged skin;
iii. stimulate an increase in one or more of Kallikrein gene in skin to which the anti-aging catalyst composition is applied followed by application of a skin-care composition comprising at least ascorbic acid or a derivative thereof, ferulic acid and tocopherol;
iv. or combinations thereof.

8. The anti-aging catalyst composition according to claim 1, wherein the anti-aging catalyst composition includes any one or combination of pH adjusters, chelating agents, skin actives, humectants, skin penetrating agents, antioxidants, plant extracts, plant oils and butters, fragrances, fillers, powders, clays, pearlescent agents, odor absorbers, coloring materials and dyes, essential oils, vitamins, antimicrobials and preservatives, nature based oils, synthetic oils, silicone oils, hydrocarbon based oils, waxes, surfactants, emulsifiers, or a combination thereof.

9. The anti-aging catalyst composition according to claim 1, wherein the anti-aging catalyst composition excludes any one or combination of pH adjusters, chelating agents, skin actives, humectants, skin penetrating agents, antioxidants, plant extracts, plant oils and butters, fragrances, fillers, powders, clays, pearlescent agents, odor absorbers, coloring materials and dyes, essential oils, vitamins, antimicrobials and preservatives, nature based oils, synthetic oils, silicone oils, hydrocarbon based oils, waxes, surfactants, emulsifiers, or a combination thereof.

10. The anti-aging catalyst composition according to claim 1, wherein the anti-aging catalyst composition excludes any one or combination of ascorbic acid and other forms of vitamin C, ferulic acid, gallic acid, zinc PCA, azelaic acid, chlorogenic acid, and vitamin E, parabens, phthalates, cyclomethicones, silicone, mineral oil, petrol based ingredients, synthetic dyes, sulfates, polyquaternium, microplastics, EDTA, silicone oils, mineral UV filter agents, organic UV filter agents, or a combination thereof.

11. The anti-aging catalyst composition according to claim 1, wherein the anti-aging catalyst composition is in the form of a serum, emulsion, or gel.

12. An anti-aging catalyst composition comprising:
a blend of cosmetically acceptable acids comprising at least one beta hydroxy acid and at least three alpha-hydroxy acids, and one or more other cosmetically acceptable acids, wherein the blend of cosmetically acceptable acids includes at least salicylic acid, glycolic acid, lactic acid, mandelic acid and phytic acid, the blend of cosmetically acceptable acids present in the composition in a total amount of 7.7%, by weight, based on the total weight of anti-aging catalyst composition;
at least one amino acid comprising taurine, and
a cosmetically acceptable solvent comprising at least water,
wherein the pH of the skin care composition is in a range from about pH 3.6 to about pH 3.9.

13. A method of modulating, ameliorating, reducing, or reversing the manifestation of the dermatological signs of chronologically aged, hormonally aged, or photo-aged skin, comprising: administering to the skin a cosmetically, dermatologically, pharmaceutically, or physiologically effective amount of the anti-aging catalyst composition according to claim 1;
the effective amount including an amount sufficient to:
i. up-regulate the expression level of at least one gene associated with regulating cell division and at least one gene associated with promoting epidermal differentiation;
ii. down-regulate the expression level of at least one gene associated with cornification, epidermis development, and keratinization; or
iii. a combination thereof.

14. The method according to claim 13, wherein the treatment with the anti-aging catalyst composition is followed by treatment of the skin with an anti-aging skin care composition or treatment regimen that includes one or more composition ingredients selected from the group consisting of antioxidants, Vitamin C, vitamin E, retinol, niacinamide, panthenol, glycerin, squalane, ceramides, collagen, hyaluronic acid, peptides, plant and fruit extracts, and sun filters, and combinations thereof.

15. The method according to claim 14, wherein the anti-aging skin care composition or treatment regimen includes a skin care composition comprising at least ascorbic acid or a derivative thereof, ferulic acid and tocopherol, and wherein the anti-aging catalyst composition includes taurine, and the blend of cosmetically acceptable acids in the anti-aging catalyst composition includes at least salicylic acid, glycolic acid, lactic acid, mandelic acid and phytic acid, the blend of cosmetically acceptable acids present in the composition in a total amount of 7.7%, by weight, based on the total weight of anti-aging catalyst composition.
